(19) United States
(12) Plant Patent Application Publication
(10) Pub. No.: US 2016/0205823 P1
(43) Pub. Date: Jul. 14, 2016

(54) Corylus plant name 'McDonald'

(50) ;

(71) Applicant: Oregon State University, Corvallis, OR (US)

(72) Inventors: Shawn A. Mehlenbacher, Corvallis, OR (US); David C. Smith, Corvallis, OR (US); Rebecca L. McCluskey, Corvallis, OR (US)

(73) Assignee: Oregon State University, Corvallis, OR (US)

(21) Appl. No.: 14/544,504

(22) Filed: Jan. 13, 2015

(51) Int. Cl. A01H 5/00

(52) U.S. Cl. PLT/152

(57) ABSTRACT

A new and distinct Corylus plant named ‘McDonald’ characterized by moderate vigor and upright-spreading plant habit, yellowish-green developing and fully expanded leaves during the spring and summer, resistance to eastern filbert blight caused by the fungus Anisogramma anomala (Peck) E. Müller, presence of random amplified polymorphic DNA markers 152-800 and 268-580 in DNA, expression of incompatibility alleles S₂ and S₁₅ in the styles, and DNA fingerprints at 14 of 21 microsatellite marker loci differ from both parents, ‘Tonda Pacifica’ and ‘Santiam’, and from one parent at an additional 5 marker loci.

### ACKNOWLEDGMENT OF GOVERNMENT SUPPORT

This invention was made with government support under Specific Cooperative Agreement No. 58-5358-4542 awarded by the United States Department of Agriculture. The government has certain rights in the invention.

### BOTANICAL DENOMINATION

Corylus avellana cultivar

### VARIETY DESIGNATION

‘McDonald’

### BACKGROUND

The present Invention relates to a new and distinct cultivar of Corylus plant, botanically known as Corylus avellana, and hereinafter referred to by the name ‘McDonald’.

The new Corylus resulted from a controlled cross of female parent ‘Tonda Pacifica’ (U.S. Plant Pat. No. 22,715) and male parent ‘Santiam’ (unpatented) made in 1997. Hybrid seeds from the cross were harvested in August 1997, stratified, and seedlings grown in the greenhouse during the summer of 1998. From this cross, a total of 132 seedling trees were planted in the field in Corvallis in October, 1998. ‘McDonald’ was discovered and selected as a single plant within the progeny of the stated cross-pollination in a controlled environment in Corvallis, Oreg., USA. It was originally assigned the designation OSU 880.027, which indicates the row and tree location of the original seedling.

‘Tonda Pacifica’ (OSU 228.084) was released in 2010 (Mehlenbacher et al., 2011), is from a cross of ‘Tonda Gentile delle Langhe’×OSU 23.024 (=‘Barcelona’×‘Extra Ghiaghli’). ‘Santiam’ (Mehlenbacher et al., 2007), released by the Oregon Agricultural Experiment Station in 2005, carries a dominant allele for a very high level of resistance to eastern filbert blight (EFB) from ‘Gasaway’ (unpatented). ‘Tonda Gentile delle Langhe’ (unpatented), the female parent of ‘Tonda Pacifica’, is an important cultivar in Piemonte, northern Italy. ‘Barcelona’ (unpatented), Oregon's most widely planted hazelnut cultivar, is known as ‘Castanyera’ where it originated in Catalunya, Spain. ‘Extra Ghiaghli’ (unpatented), obtained from Greece, is a clone of the important Turkish cultivar ‘Tombul’. ‘Gasaway’ (unpatented) is the source of a single dominant gene for resistance to eastern filbert blight.

The new cultivar was asexually reproduced by rooted suckers annually for nine years (2004-2005, 2007-2013). The new cultivar was also asexually propagated by whip grafting in 2005. The unique features of this new Corylus are stable and reproduced true-to-type in successive generations of asexual reproduction.

### SUMMARY

The following traits have been observed and are determined to be the unique characteristics of ‘McDonald’. These characteristics in combination distinguish ‘McDonald’ as a new and distinct cultivar:

1. Moderate vigor and upright-spreading plant habit.

2. Yellowish-green developing and fully expanded leaves during the spring and summer.

3. Resistance to eastern filbert blight caused by the fungus Anisogramma anomala (Peck) E. Müller.

4. Presence of random amplified polymorphic DNA markers 152-800 and 268-580 in DNA of ‘McDonald’ amplified by the polymerase chain reaction. These two markers are linked to a dominant allele for resistance to eastern filbert blight from the cultivar ‘Gasaway’ (unpatented).

5. Expression of incompatibility alleles S₂ and S₁₅ in the styles.

6. DNA fingerprints at 14 of 21 microsatellite marker loci differ from both parents, ‘Tonda Pacifica’ and ‘Santiam’, and from one parent at an additional 5 marker loci. Additional DNA fingerprints of grandparent ‘Tonda Gentile delle Langhe’, standard cultivar ‘Barcelona’, eastern filbert blight resistance source ‘Gasaway’ and five cultivars released by Oregon State University hazelnut breeding program are shown in Table 6.

Comparisons in two replicated trials conducted in Corvallis, Oreg., plants of the new Corylus differed from plants of the Corylus avellana cultivar ‘Barcelona’ and other cultivars and selections of Corylus avellana known to the Inventors primarily in nut size, nut shape, kernel percentage (ratio of kernel weight to nut weight), frequency of defects (blank nuts, moldy kernels, twins, etc.), time of pollen shed, time of nut maturity, length of the husk or involucre, and plant size.

‘McDonald’ is well-suited to the blanched kernel market for use in chocolate products and baked goods. ‘McDonald’ combines high nut yield, early nut maturity, small round nuts and kernels, high kernel percentage, good kernel blanching and excellent flavor. The tree is moderately vigorous and has a desirable growth habit.

DNA markers and field observations indicate that ‘McDonald’ has a very high level of resistance to eastern filbert blight (EFB) caused by the fungus Anisogramma anomala (Peck) E. Müller. The resistance is conferred by a dominant allele from ‘Gasaway’. EFB is now present throughout the Willamette Valley where 99% of the U.S. hazelnut crop is grown. Pruning to remove cankers and fungicide applications are currently used to manage the disease in orchards of ‘Barcelona’ and other susceptible cultivars. Thus, ‘McDonald’ is suitable for planting in areas with high disease pressure, as are previous releases ‘Santiam’ (2005), ‘Yamhill’ (2008, unpatented), ‘Jefferson’ (2009, unpatented), ‘Dorris’ (2012, U.S. Plant Pat. No. 25,022) and ‘Wepster’ (2013, U.S. application Ser. No. 13/998,648). ‘Wepster’, released as a cultivar in 2013, is cross-compatible with ‘McDonald’ in both directions. Thus ‘Wepster’ and ‘McDonald’ can be planted together with each serving as a pollinizer for the other.

### BRIEF DESCRIPTION OF THE DRAWINGS

The accompanying colored photographs illustrate the overall appearance of the new cultivar, showing the colors as true as it is reasonably possible to obtain in colored reproductions of this type. Foliage colors in the photographs may differ slightly from the color values cited in the detailed botanical description which accurately describe the colors of the new Corylus.

FIG. 1 shows a tree the new cultivar ‘McDonald’ hazelnut in the 8th leaf.

FIG. 2 shows a tree of the new cultivar ‘McDonald’ hazelnut in winter at end of 7th leaf.

FIG. 3 shows a tree of the new cultivar ‘McDonald’ tree in winter at end of 7th leaf.

FIG. 4 shows August 2013 trees of the new cultivar ‘McDonald’ (left) and ‘Jefferson’ (right, unpatented), 8th leaf.

FIG. 5 shows trees of the new cultivar ‘McDonald’ (left) and ‘Wepster’ (right, unpatented), 8th leaf.

FIG. 6 shows nuts, husks and leaves of ‘McDonald’ hazelnut.

FIG. 7 shows nuts and husks of ‘McDonald’ hazelnut.

FIG. 8 shows nuts, raw kernels, and blanched kernels of hazelnuts ‘Wepster’ (OSU 894.030), ‘Yamhill’, ‘McDonald’ (OSU 880.027) and ‘Barcelona’.

FIG. 9 shows nuts, raw kernels and blanched kernels of hazelnuts ‘Yamhill’, ‘McDonald’ (OSU 880.027) and ‘Barcelona’.

FIG. 10 shows nuts, raw kernels and blanched kernels of hazelnuts ‘McDonald’ (OSU 880.027) and ‘Barcelona’.

FIG. 11 is a graph showing maturity of ‘McDonald’ (OSU 880.027) and ‘Barcelona’ based on percentage of nuts on the ground. Four trees of each genotype were harvested in 2013 on four dates (September 13, September 20, September 27 and October 4). Percentages of nuts on the ground based on weight are cumulative.

FIGS. 12A-12B show a phenology chart showing time of female receptivity (bottom, red) and pollen shed (top, green) of ‘McDonald’ (OSU 880.027) and other hazelnut cultivars (Dec 2011-Mar 2012).

FIGS. 13A-13D show a phenology chart showing bloom stage and pollen shed of hazelnut cultivars, pollinizers and selections (Dec 2012-Mar 2013).

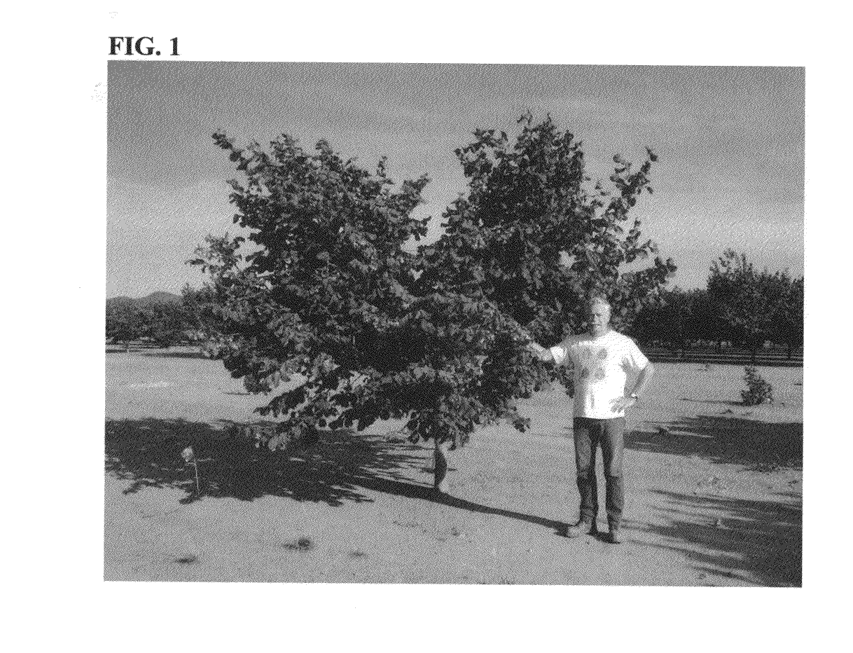

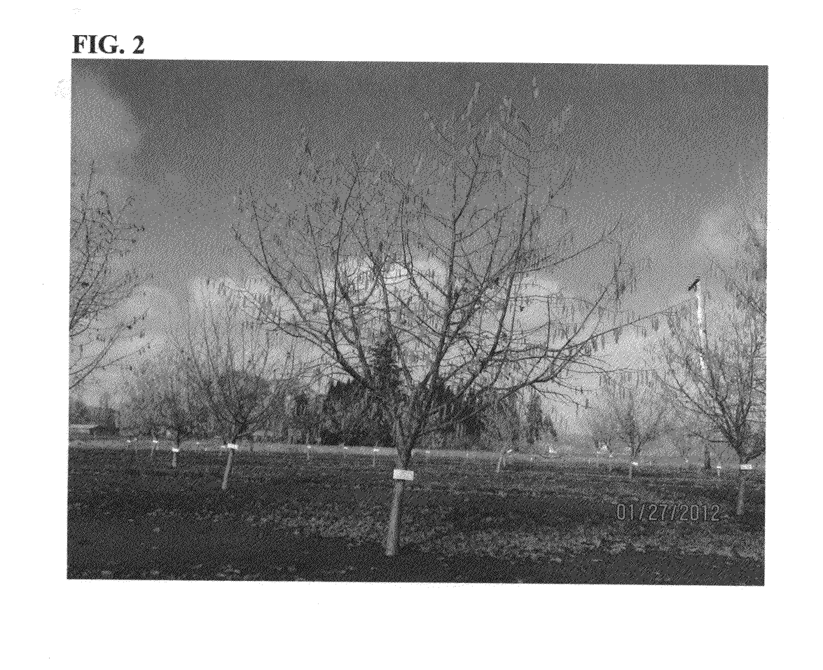

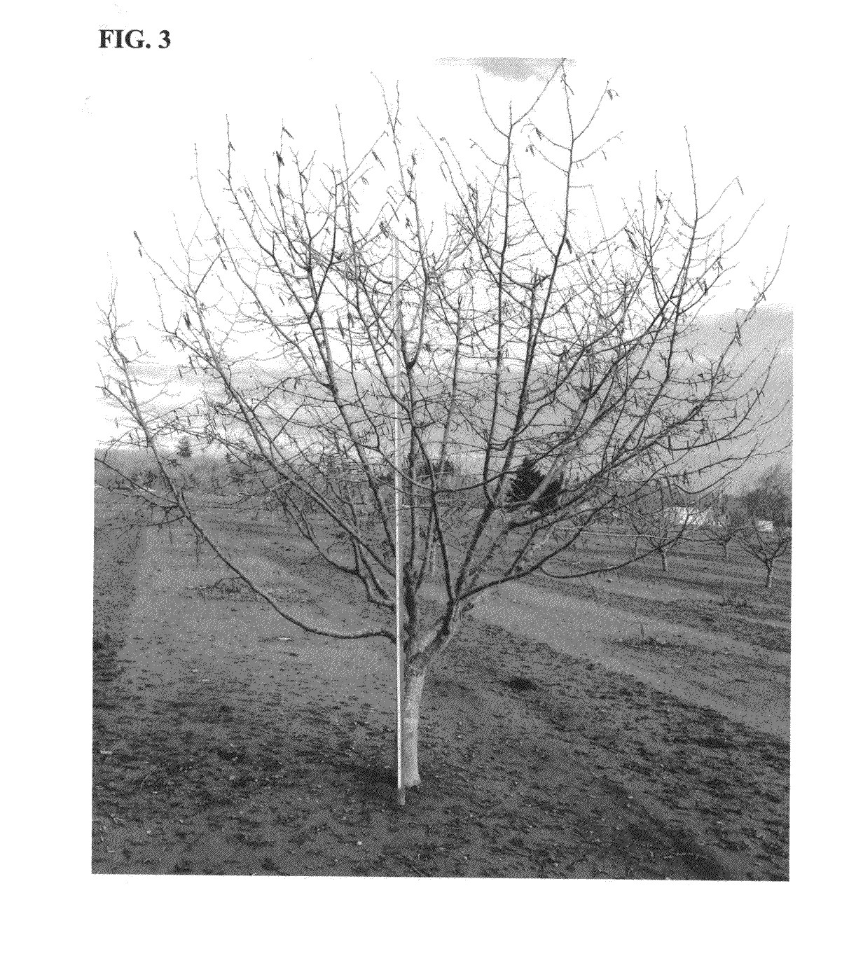

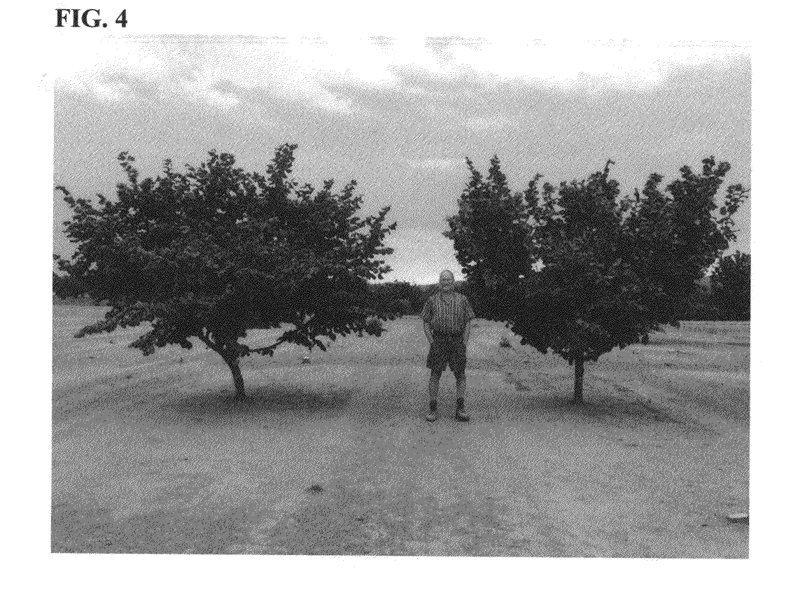

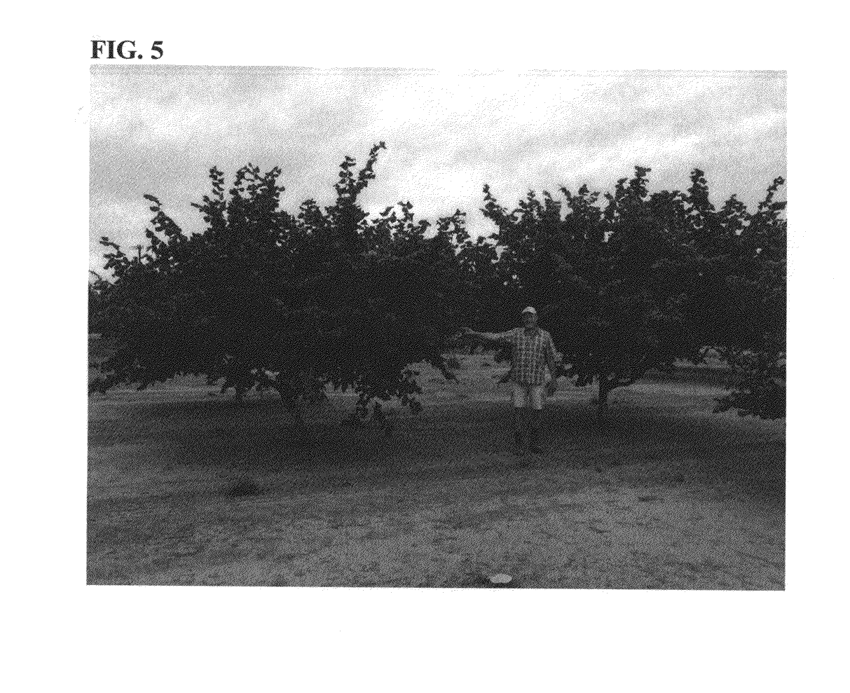

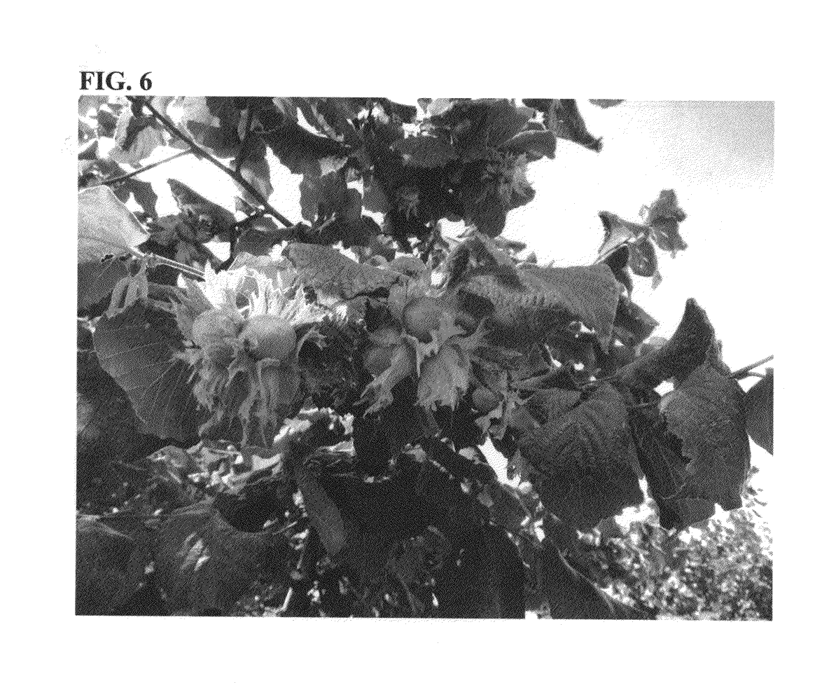

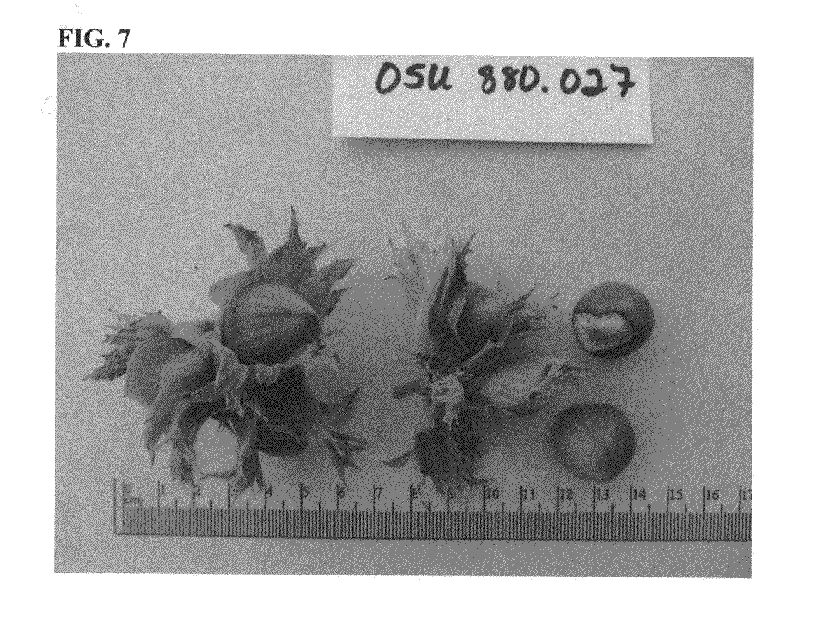

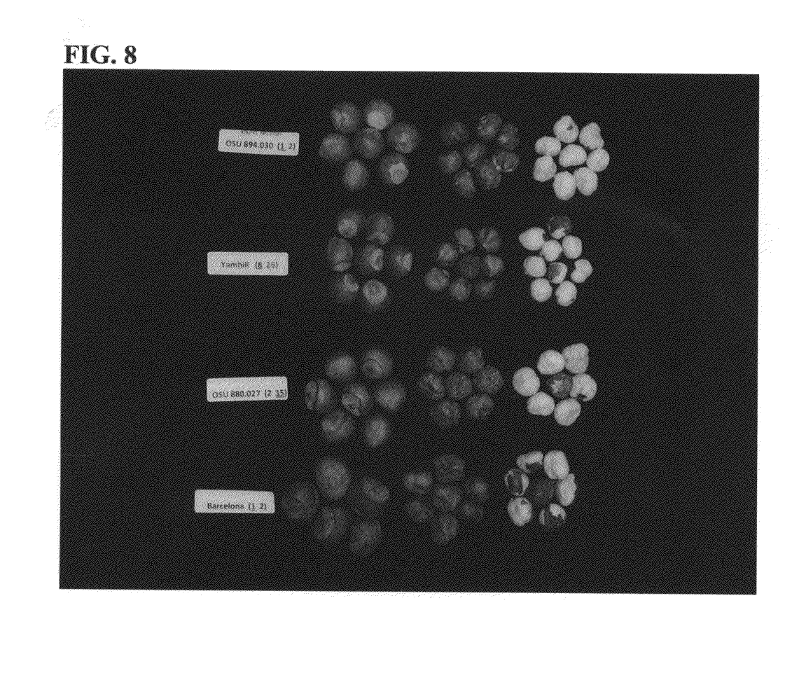

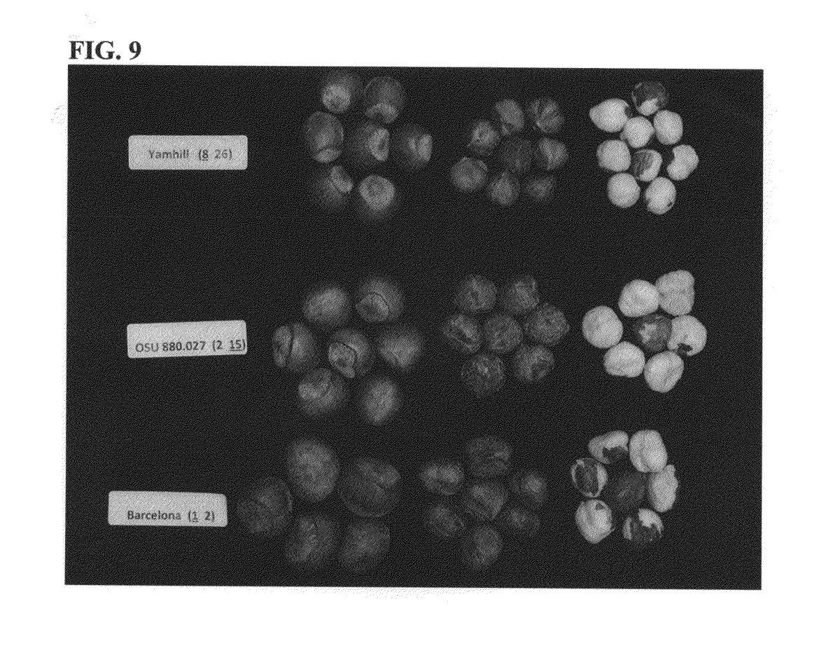

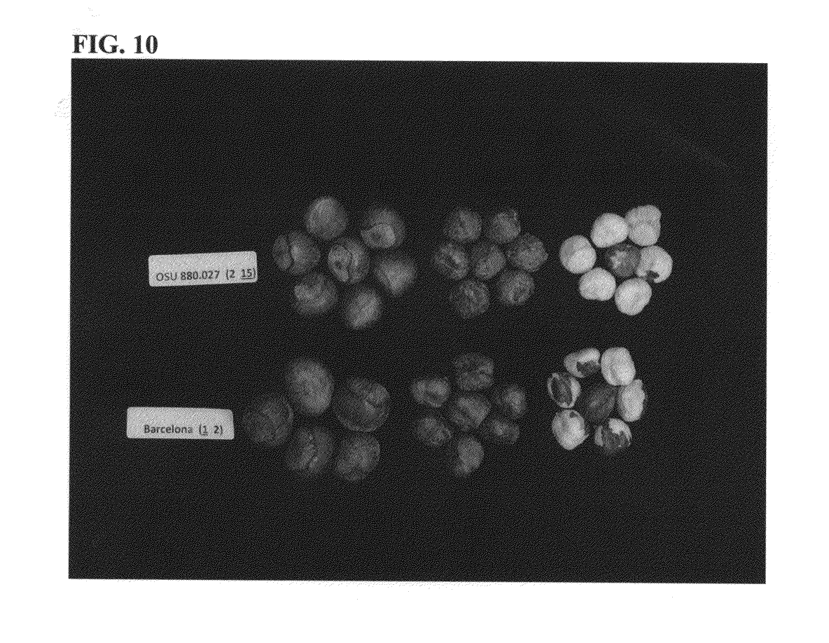

### DETAILED DESCRIPTION

The cultivar ‘McDonald’ has not been observed under all possible environmental conditions. The phenotype may vary somewhat with variations in environment such as temperature and light intensity, without, however, any variance in genotype. The aforementioned photographs and following observations and measurements describe plants grown in Corvallis, Oreg. under commercial practice outdoors in the field during the fall, winter and spring. Plants used for the photographs and description were propagated by tie-off layerage and growing on their own roots, and seven or eight years old. In the following description, color references are made to The Royal Horticultural Society Colour Chart, 1966 Edition, except where general terms of ordinary dictionary significance are used.
Botanical classification: Corylus avellana cultivar ‘McDonald’.
Parentage:
   Female, or seed, parent.—Corylus avellana cultivar ‘Tonda Pacifica’ (U.S. Plant Pat. No. 22,715).
   Male, or pollen, parent.—Corylus avellana cultivar ‘Santiam’ (unpatented).
Propagation (type rooted suckers):
   Time to initiate roots.—About 30 days at 20° C.
   Time to produce a rooted young plant.—About six months at 22° C.
   Root description.—Fine to thick; freely branching; creamy white in color.
Propagation (type whip grafting):
   Time to budbreak on the scions.—About 14 days at 25° C.
   Time to produce a grafted plant.—About six months at 25° C.
Plant description:
   General appearance.—Perennial shrub. Upright-spreading plant habit.
   Growth and branching habit.—Freely branching; about 15 lateral branches develop per plant. Pinching, that is, removal of the terminal apices, enhances branching with lateral branches potentially forming at every node.
   Vigor.—Moderate vigor growth habit.
   Size.—Plant height is about 5 meters; plant diameter or spread is about 5 meters.
Lateral branch description:
   Length.—About 33.5 cm.
   Diameter.—About 5 mm.
   Internode length.—About 3.2 cm.
   Texture.—Smooth, glabrous.
   Strength.—Strong. Color, immature: 139D.
   Color, mature.—177D.
Foliage description:
   Arrangement.—Alternate, simple.
   Length.—About 10.4 cm.
   Width.—About 8.7 cm.
   Shape.—Oblong to ovate.
   Apex.—Obtuse to acute.
   Base.—Cordate.
   Margin.—Serrate.
   Texture, upper and lower surfaces.—Slightly pubescent.
   Venation pattern.—Pinnate.
   Color.—Developing foliage, upper surface 141C, lower surfaces: 139C. Fully expanded foliage, upper surface: Spring and summer, 139B; late summer and fall, 139B. Fully expanded foliage, lower surface: Spring and summer, 139C; late summer and fall, 139C. Venation, upper surface: Spring and summer, 139C; late summer and fall, 139C. Venation, lower surface: Spring and summer, 139D; late summer and fall, 139D.
Petiole description:
   Length.—About 2.7 cm.
   Diameter.—About 1.8 mm.
   Texture, upper and lower surfaces.—Pubescent.
   Color, upper surface.—Spring and summer, 139D; late summer and fall, 139D.
   Color, lower surface.—Spring and summer, 139D; late summer and fall, 139D.
Flower description: Male inflorescences are catkins, color prior to elongation 176D. Female inflorescence style color 047B.
Nut description:
   Length.—About 18.4 mm.
   Width.—About 18.9 mm.
   Depth.—About 17.9 mm.
   Nut shape.—Round. Nut shape index [(Width+Depth)/2*Length]=1.00. Nut compression index (Width/Depth)=1.05.
   Nut shell color.—164A. Nut weight: About 2.51 grams.
   Kernel weight.—About 1.29 grams.
   Kernel percentage (kernel weight/nut weight).—About 51.5%.
Disease/pest resistance: Plants of the new Corylus are highly resistant to eastern filbert blight caused by the fungus Anisogramma anomala (Peck) E. Müller, although a few small cankers may develop under high disease pressure. Plants of the new Corylus are resistant to bud mites (Phytoptus avellanae Nal.), while plants of ‘Tonda Gentile delle Langhe’ are highly susceptible, and plants of ‘Barcelona’ are highly resistant.
Temperature tolerance: Plants of the new Corylus have been observed to tolerate temperatures from −21 to 38° C. in the field in Corvallis, Oreg.

### Comparative Data:

Tree size, growth habit, yield, and yield efficiency.

Tree sizes in the two trials were estimated by measuring trunk diameters 30 cm above the soil line, at the end of the 7^{th} growing season (December 2012 and 2013, respectively). Trunk cross-sectional area (TCA) was calculated from trunk diameter. Trees of ‘McDonald’ are moderately vigorous, and their upright-spreading growth habit should be grower-friendly (easy to manage).

In the first trial (Table 1), TCA of ‘McDonald’ (86.9 cm²) was similar to ‘Jefferson’ (unpatented), ‘Dorris’, and ‘York’ (U.S. Plant Pat. No. 24,972). Trees were larger than ‘Yamhill’ (unpatented) but smaller than ‘Wepster’. Total nut yield per tree was 17.11 kg, which is slightly less than ‘Jefferson’, ‘Yamhill’ and ‘Wepster’ and about the same as ‘Dorris’ and ‘Sacajawea’ (unpatented). Nut yield efficiency of ‘McDonald’ (0.198 kg/cm²) was similar to ‘Sacajawea’ and ‘Wepster’, and lower than ‘Jefferson’ and ‘Yamhill’.

In the second trial (Table 1), trees of ‘McDonald’ were similar in size to ‘Jefferson’. In adjacent rows planted at the same time to EFB-susceptible selections, TCAs for ‘Barcelona’, ‘Lewis’ and ‘Clark’ were 138.6, 77.6 and 63.7 cm², respectively. The TCA of ‘McDonald’ is 63% of ‘Barcelona’. In previous trials, tree size of ‘Jefferson’ and ‘Lewis’ has been about 70% of ‘Barcelona’. Growers have been pleased with this level of vigor. Total nut yield per tree was 21.43 kg for ‘McDonald’ vs. 22.99 kg for ‘Jefferson’, 16.88 kg for the pollinizer ‘Felix’ (U.S. Plant Pat. No. 24,973), and 17.68 kg for ‘Santiam’. Nut yield efficiency for ‘McDonald’7 (0.245 kg/cm²), which adjusts for differences in tree size, was similar to ‘Santiam (0.267 kg/cm²), lower than ‘Jefferson’ (0.299 kg/cm²) and higher than ‘Felix’ (0.133 kg/cm²).

TABLE 1
Nut yield, trunk cross-sectional area, and yield efficiency of
‘McDonald’ and other hazelnut cultivars and
selections in two trials

| | No. | Yield per tree (kg) | | | |
|---|---|---|---|---|---|
| Cultivar | trees | Year 3 | Year 4 | Year 5 | Year 6 |

First trial (2006 planting)

| | | | | | |
|---|---|---|---|---|---|
| OSU 833.082 | 7 | 0.71 | 2.60 | 2.25 | 5.95 |
| OSU 879.031 | 7 | 0.31 | 1.42 | 2.13 | 5.17 |
| ‘McDonald’ | 7 | 0.29 | 1.13 | 2.44 | 6.54 |
| OSU 881.078 | 7 | 0.23 | 1.08 | 2.08 | 5.08 |
| Dorris | 7 | 0.42 | 2.84 | 3.30 | 6.51 |
| Jefferson | 7 | 0.41 | 3.55 | 3.35 | 6.97 |
| Sacajawea | 7 | 0.26 | 1.92 | 2.51 | 6.52 |
| Santiam | 7 | 0.29 | 1.76 | 3.73 | 7.25 |
| Wepster | 7 | 0.24 | 1.56 | 2.56 | 6.62 |
| Yamhill | 7 | 0.78 | 2.79 | 3.88 | 7.34 |
| York | 7 | 0.41 | 1.52 | 2.48 | 6.13 |
| LSD _{0.05} | | 0.17 | 0.55 | 0.75 | 0.89 |

Second trial (2007 planting)

| | | | | | |
|---|---|---|---|---|---|
| ‘McDonald’ | 4 | 0.15 | 1.10 | 4.85 | 7.38 |
| Felix | 4 | 0.06 | 1.04 | 2.91 | 7.93 |
| Jefferson | 4 | 0.55 | 1.97 | 5.63 | 4.60 |
| Santiam | 4 | 0.20 | 1.11 | 4.09 | 5.46 |
| LSD _{0.05} | | 0.21 | 0.43 | 0.54 | 2.04 |

| | Yield per tree (kg) | | TCA^{z} | YE^{y} |
|---|---|---|---|---|
| Cultivar | Year 7 | Total | (cm²) | (kg · cm⁻²) |

First trial (2006 planting)

| | | | | |
|---|---|---|---|---|
| OSU 833.082 | 2.46 | 13.97 | 68.1 | 0.205 |
| OSU 879.031 | 6.54 | 15.58 | 87.2 | 0.179 |
| ‘McDonald’ | 6.71 | 17.11 | 86.9 | 0.198 |
| OSU 881.078 | 6.67 | 15.15 | 112.4 | 0.135 |
| Dorris | 5.70 | 18.78 | 84.0 | 0.225 |
| Jefferson | 5.79 | 20.07 | 85.4 | 0.235 |
| Sacajawea | 6.76 | 17.97 | 93.7 | 0.196 |
| Santiam | 6.34 | 19.36 | 79.2 | 0.244 |
| Wepster | 8.68 | 19.67 | 99.2 | 0.198 |
| Yamhill | 4.94 | 19.73 | 78.5 | 0.249 |
| York | 4.42 | 14.97 | 85.6 | 0.175 |
| LSD _{0.05} | 1.26 | 2.62 | 10.4 | 0.027 |

Second trial (2007 planting)

| | | | | |
|---|---|---|---|---|
| ‘McDonald’ | 7.95 | 21.43 | 87.55 | 0.245 |
| Felix | 4.95 | 16.88 | 128.43 | 0.133 |
| Jefferson | 10.25 | 22.99 | 77.52 | 0.299 |
| Santiam | 6.83 | 17.68 | 66.81 | 0.267 |
| LSD _{0.05} | 1.18 | 2.45 | 13.48 | 0.029 |

^{z}Trunk cross-sectional area calculated from trunk diameters measured in late fall at the end of the seventh season.
^{y}Yield efficiency = Total nut yield/TCA.

A high percentage of the nuts and kernels of ‘McDonald’ in both trials were marketable (Table 2). Very few moldy kernels were observed in ‘McDonald’, in striking contrast to ‘Santiam’.

TABLE 2
Frequency of good nuts, and of nut and kernel defects in
‘McDonald; and other hazelnut cultivars
and selections in trials planted in 2006 and 2007

| | Frequency (%)z | | | |
|---|---|---|---|---|
| Selection | Good | Blanks | Brown stain | Moldy |

First trial (n = 7)

| | | | | |
|---|---|---|---|---|
| OSU 879.031 | 92.7 | 3.7 | 0.4 | 1.0 |
| ‘McDonald’ | 88.0 | 2.7 | 0.0 | 1.0 |
| OSU 881.078 | 84.7 | 8.9 | 0.0 | 2.1 |
| OSU 833.082 | 87.0 | 4.4 | 0.0 | 4.0 |
| Dorris | 80.7 | 7.5 | 0.2 | 4.2 |
| Jefferson | 84.2 | 3.8 | 0.1 | 4.4 |
| Sacajawea | 87.8 | 3.1 | 0.0 | 3.4 |
| Wepster | 86.7 | 7.6 | 0.2 | 1.1 |
| Yamhill | 91.3 | 2.3 | 0.1 | 2.2 |
| York | 83.7 | 8.7 | 0.3 | 1.7 |
| Santiam | 76.2 | 3.0 | 0.0 | 7.9 |
| LSD 0.05 | 5.6 | 3.6 | 0.9 | 2.2 |

Second trial (n = 4)

| ‘McDonald’ | 83.5 | 5.1 | 0.1 | 2.1 |
|---|---|---|---|---|
| Felix | 88.9 | 4.2 | 0.2 | 2.1 |
| Jefferson | 80.1 | 4.3 | 0.3 | 5.7 |
| Santiam | 68.8 | 2.8 | 0.1 | 17.3 |
| LSD 0.05 | 3.5 | 2.5 | 0.3 | 2.3 |
| | Frequency (%)z | | | |
| Selection | Shrivel | Poor fill | Twins | Black tips |

First trial (n = 7)

| | | | | |
|---|---|---|---|---|
| OSU 879.031 | 1.0 | 0.6 | 0.3 | 0.3 |
| ‘McDonald’ | 7.5 | 0.5 | 0.0 | 0.3 |
| OSU 881.078 | 2.8 | 1.1 | 0.1 | 0.3 |
| OSU 833.082 | 2.8 | 0.7 | 0.5 | 0.6 |
| Dorris | 4.3 | 1.9 | 0.1 | 1.2 |
| Jefferson | 2.8 | 3.8 | 0.5 | 0.5 |
| Sacajawea | 3.1 | 1.2 | 0.1 | 1.3 |
| Wepster | 2.7 | 1.4 | 0.1 | 0.3 |
| Yamhill | 2.5 | 1.6 | 0.0 | 0.1 |
| York | 2.8 | 1.2 | 0.0 | 1.6 |
| Santiam | 9.4 | 2.3 | 0.2 | 0.9 |
| LSD 0.05 | 2.7 | 2.3 | 0.5 | 0.9 |

Second trial (n = 4)

| | | | | |
|---|---|---|---|---|
| ‘McDonald’ | 4.5 | 4.5 | 0.1 | 0.3 |
| Felix | 0.4 | 2.9 | 0.3 | 1.1 |
| Jefferson | 0.4 | 8.9 | 0.6 | 0.6 |
| Santiam | 1.8 | 9.6 | 0.1 | 0.1 |
| LSD 0.05 | 1.0 | 2.5 | 0.4 | 0.5 |

zMeans of years 4-7.
LSD = least significant difference.

Percent kernel (the ratio of kernel weight to nut weight) for ‘McDonald’ (52%) is higher than ‘Barcelona’ (typically 43%) and ‘Jefferson’ (45%) (Table 3). Yields of kernels per acre would be high for ‘McDonald’. Although ‘McDonald’ is not the highest-yielding selection, its yields have been consistently good and the nuts consistently well-filled. In contrast, ‘Yamhill’ trees occasionally set very heavy nut crops, and the nuts can be so poorly filled that the kernels are not marketable.

TABLE 3
Nut and kernel weight, kernel percentage, and ratings for fiber,
blanching and bud mite susceptibility for ‘McDonald’ and other
hazelnut cultivars and selections in two trials.

| | | | | | | |
|---|---|---|---|---|---|---|
| | Nut | Kernel | Kernel | | | |
| | wt | wt | percen- | | Blan- | Bud |
| Selection | (g)^{z} | (g) | tage | Fiber^{y} | ching^{x} | mite^{w} |

First trial (n = 7)

| | | | | | | |
|---|---|---|---|---|---|---|
| OSU 879.031 | 2.34 | 1.15 | 49.0 | — | 5.2 | — |
| McDonald’ | 2.39 | 1.21 | 50.7 | — | 3.8 | — |
| OSU 881.078 | 2.37 | 0.97 | 41.0 | — | 4.5 | — |
| OSU 833.082 | 2.72 | 1.12 | 41.3 | — | 2.6 | — |
| Dorris | 3.24 | 1.32 | 40.8 | — | 2.9 | — |
| Jefferson | 3.53 | 1.51 | 42.8 | — | 4.8 | — |
| Sacajawea | 2.52 | 1.29 | 51.2 | — | 2.8 | — |
| Santiam | 2.09 | 1.03 | 49.5 | — | 5.1 | — |
| Wepster | 2.23 | 0.98 | 43.9 | — | 3.1 | — |
| Yamhill | 2.18 | 1.01 | 46.4 | — | 5.0 | — |
| York | 2.59 | 1.12 | 43.4 | — | 4.8 | — |
| LSD _{0.05} | 0.23 | 0.13 | 3.0 | — | 0.7 | — |

Second trial (n = 4)

| | | | | | | |
|---|---|---|---|---|---|---|
| ‘McDonald’ | 2.62 | 1.37 | 52.3 | 2.6 | 3.3 | 1.8 |
| Felix | 2.71 | 1.37 | 50.8 | 3.0 | 2.2 | 2.0 |
| Jefferson | 3.76 | 1.67 | 44.5 | 3.0 | 4.3 | 1.2 |
| Santiam | 2.28 | 1.15 | 50.6 | 3.0 | 4.2 | 2.2 |
| LSD _{0.05} | 0.22 | 0.04 | 1.0 | 0.1 | 0.4 | 0.2 |

^{z}Means for nuts and kernels are over four years.
^{y}Amount of fiber on the pellicle was rated in the second trial from 1 (none) to 4 (much).
^{x}Blanching was rated from 1 (complete pellicle removal) to 7 (no pellicle removal).
^{w}Susceptibility to bud mite (primarily Phytoptus avellanae Nal.) was rated on four trees of each selection in the second trial on a scale of 1 (no blasted buds) to 5 (many blasted buds).
Shown are mean ratings for 5 years (2009-2013).
LSD = least significant difference.

Nut maturity date.

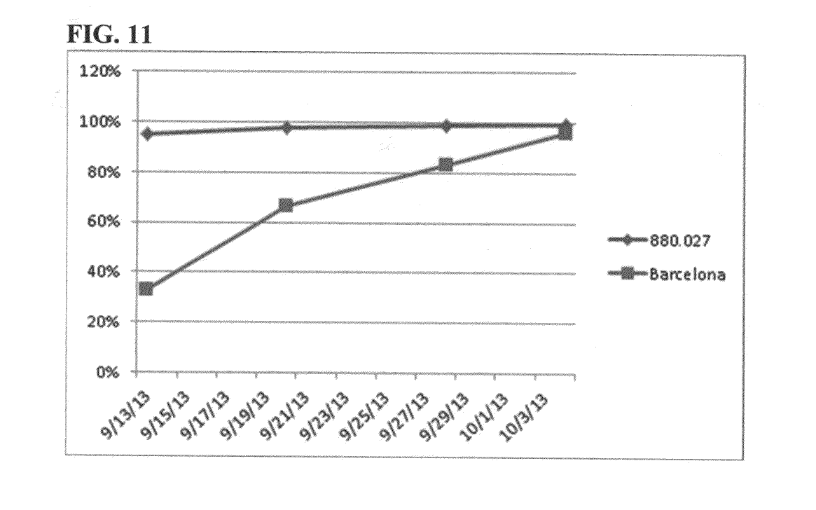

The nuts of ‘McDonald’ are borne in clusters of 2-3 in husks about 50% longer than the nuts. The husks are slit down the side, and flare open as they dry at maturity. About 95% of the nuts fall free of the husk at maturity (range 85-100%). The other 5% of the nuts would come out of the husks as they moved through the harvester. When mature, the shells are light tan in color. Harvest date is estimated to be 14 days before ‘Barcelona’, allowing it to be harvested before the start of the rainy season (Table 4 and FIG. 11).

TABLE 4
Harvest notes for four hazelnut genotypes harvested by hand
for three years in the second trial.

| Year | Selection | Date | Down^{z} | Free^{y} |
|---|---|---|---|---|
| 2010 | ‘McDonald’ | 9/28 | 88 | 99 |
| | Felix | 10/12 | 87 | 77 |
| | Jefferson | 10/12 | 86 | 78 |
| | Santiam | 9/28 | 89 | 97 |
| 2011 | ‘McDonald’ | 10/27 | 96 | 93 |
| | Felix | 10/27 | 96 | 81 |
| | Jefferson | 10/27 | 88 | 64 |
| | Santiam | 9/30 | 81 | 94 |
| 2012 | ‘McDonald’ | 10/4 | 97 | 98 |
| | Felix | 10/18 | 85 | 84 |
| | Jefferson | 10/18 | 81 | 89 |
| | Santiam | 10/5 | 97 | 91 |

^{z}Estimated percentage of nuts on the ground as opposed to in the tree.
^{y}Of the nuts on the ground, estimated percentage free of the husk.

Nuts in the first trial were annually raked and fed through a Mave harvester. Nuts in the second trial were hand-harvested and notes recorded at the time of harvest (Table 4). Notes included the date, the percentage of nuts on the ground (as opposed to in the tree), and the percentage of the nuts on the ground that were free of the husk (as opposed to inside the husk). We harvest more than 2000 trees by hand every fall, visiting a block once every 7 to 10 days, and harvesting a tree if >70% of the nuts are on the ground. Thus the percentages are estimates. The harvest dates and percentage down indicate that maturity of ‘McDonald’ is about 14 days earlier than ‘Barcelona’ (Table 4). To more precisely estimate nut maturity, the four trees of ‘McDonald’ in the second trial and four trees of ‘Barcelona’ in adjacent rows were harvested by hand at weekly intervals in the final year of the trial (2013). The maturity curves from the 2013 harvest (FIG. 11) show that nuts of ‘McDonald’ drop at least 14 days sooner than those of ‘Barcelona’. Most orchards planted since 2009 have been of ‘Jefferson’, which matures about three days after ‘Barcelona’. Plantings of ‘McDonald’ and ‘Wepster’ would allow harvest to begin two weeks earlier.

Nut and kernel characteristics:

‘McDonald’ can be used in the blanched kernel market as a companion for ‘Wepster’ and an alternative to ‘Yamhill’, ‘Lewis’, ‘Clark’ and ‘Sacajawea’. ‘McDonald’, like ‘Wepster’ and ‘Yamhill’, has a very high level of EFB resistance, while ‘Lewis’, ‘Clark’ and ‘Sacajawea’ have lower level of quantitative resistance. The nut shape is round with a slight point, which lends itself well to sizing and cracking. The shells are thin and easy to crack, and most kernels remain whole when the shell is broken. The kernel size of ‘McDonald’ is small, similar to ‘Clark’, and larger than ‘Wepster’. Raw kernels are attractive and have a light brown pellicle with a moderate amount of attached fiber (rating =2.6) on a scale of 1 (no fiber) to 4 (much fiber). Pellicle removal is rated on a scale of 1 (complete pellicle removal) to 7 (no pellicle removal), with ratings <4.0 being desired. Most of the pellicle on ‘McDonald’ kernels is removed from the kernels with dry heat in the blanching process (ratings=3.8 and 3.3 in the first and second trials, respectively) which is better than ‘Yamhill’ (rating=5.0). Kernel texture, flavor and aroma are excellent, and desirable for use in baked goods and chocolate products.

In two trials, ‘McDonald’ produced fewer nut and kernel defects (and more good kernels) than ‘Barcelona’ in adjacent rows. The nut and kernel data were similar in the two trials. The frequency of moldy kernels in ‘McDonald’ in the two trials was very low (1.0-2.1%) in contrast to ‘Jefferson’ (4.4-5.7%) and especially ‘Santiam’ (7.89-17.3%). Kernel mold is a problem in ‘Lewis’ and ‘Santiam’, particularly when weather is cool and wet in spring and early summer. The frequency of poorly filled nuts is low compared to other selections, even though ‘McDonald’ crops consistently well.

Incompatibility and pollinizers.

The trees set a moderate to high amount of catkins that shed copious amounts of pollen in early mid-season, with ‘Wepster’, ‘York’ and ‘Yamhill’. Pollen has been collected and used in several controlled pollinations, and both quantity and viability appear to be very good. ‘McDonald’ has incompatibility alleles S₂ and S₁₅ as determined by fluorescence microscopy. Both alleles are expressed in the females, but only S₁₅ is expressed in the pollen because of dominance. By convention, alleles expressed in the pollen are underlined.

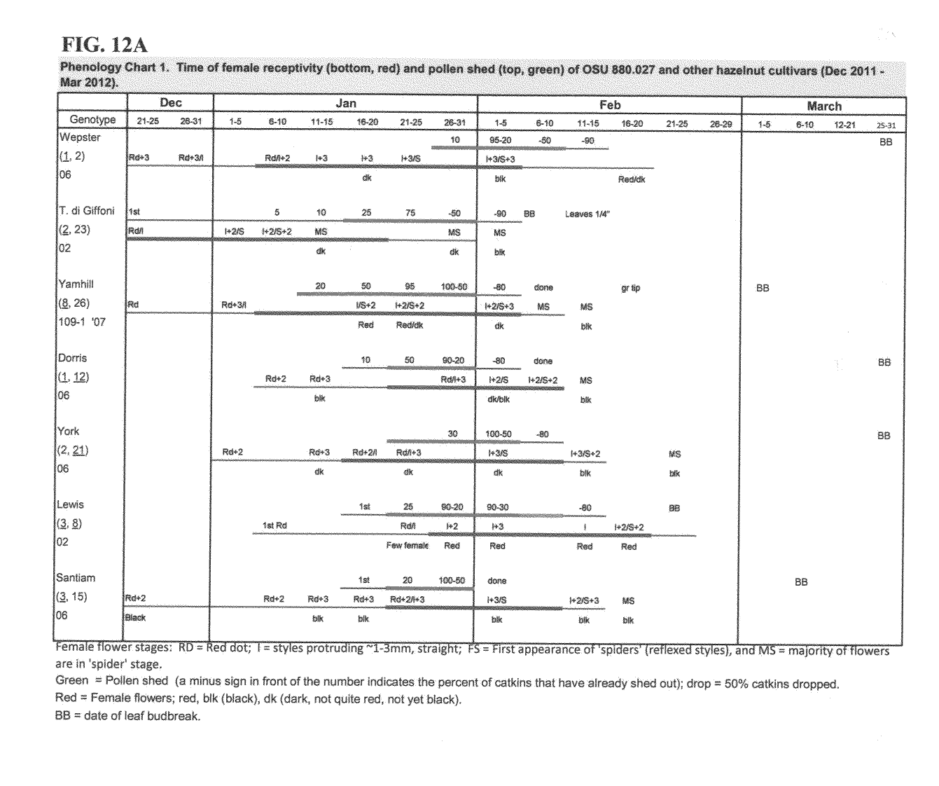

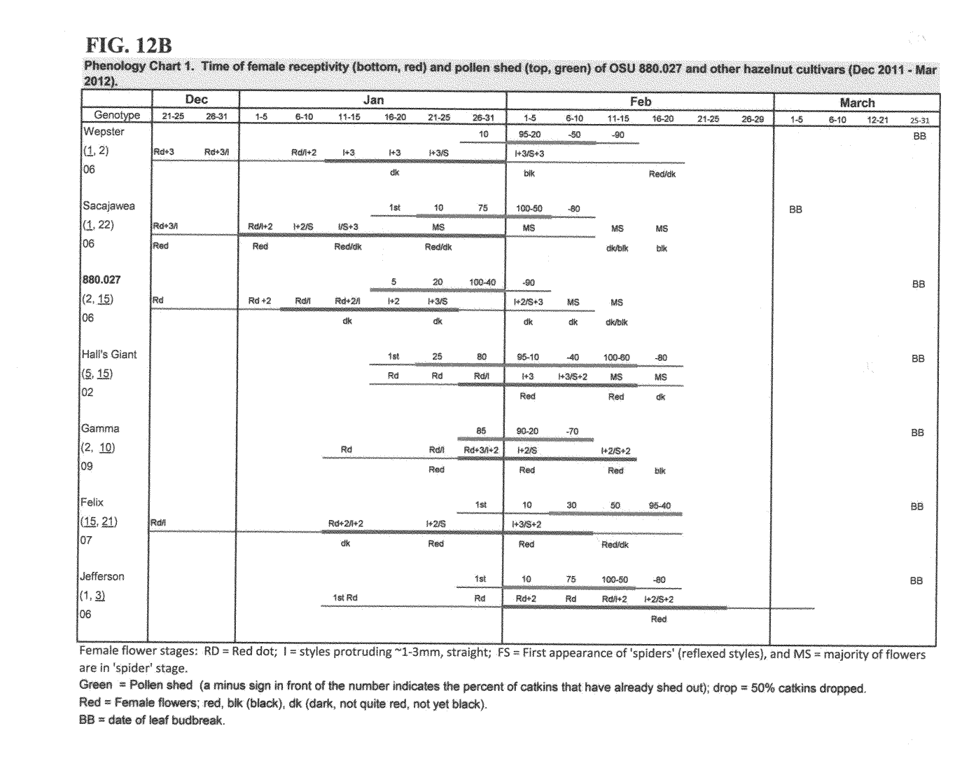

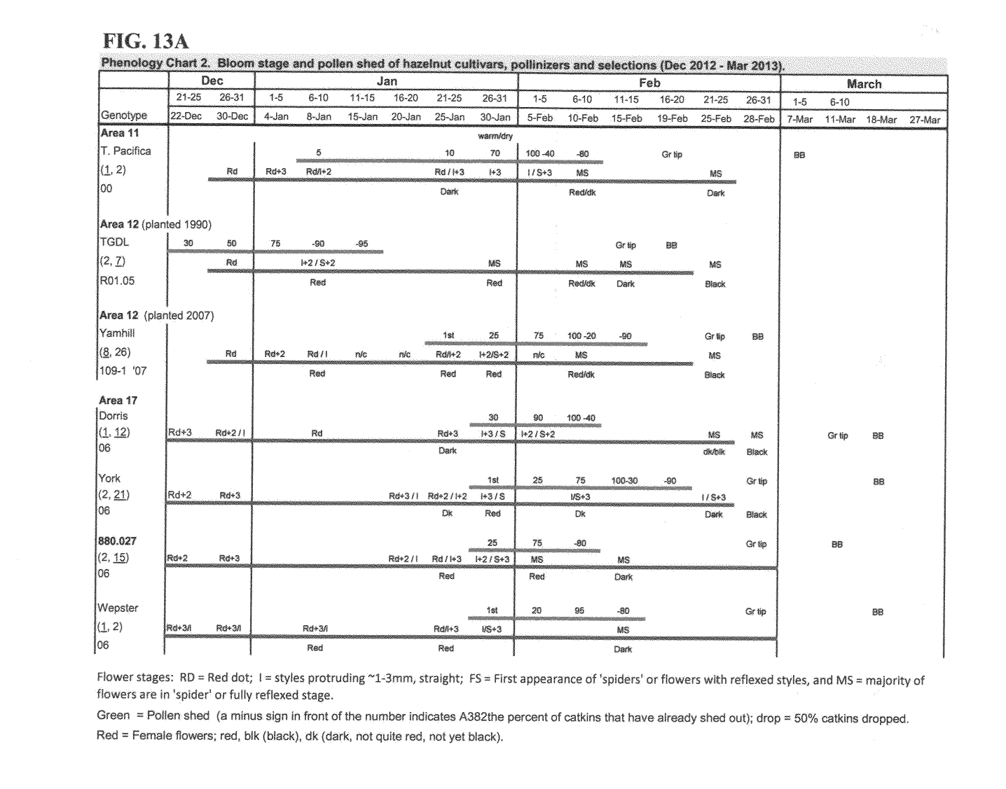

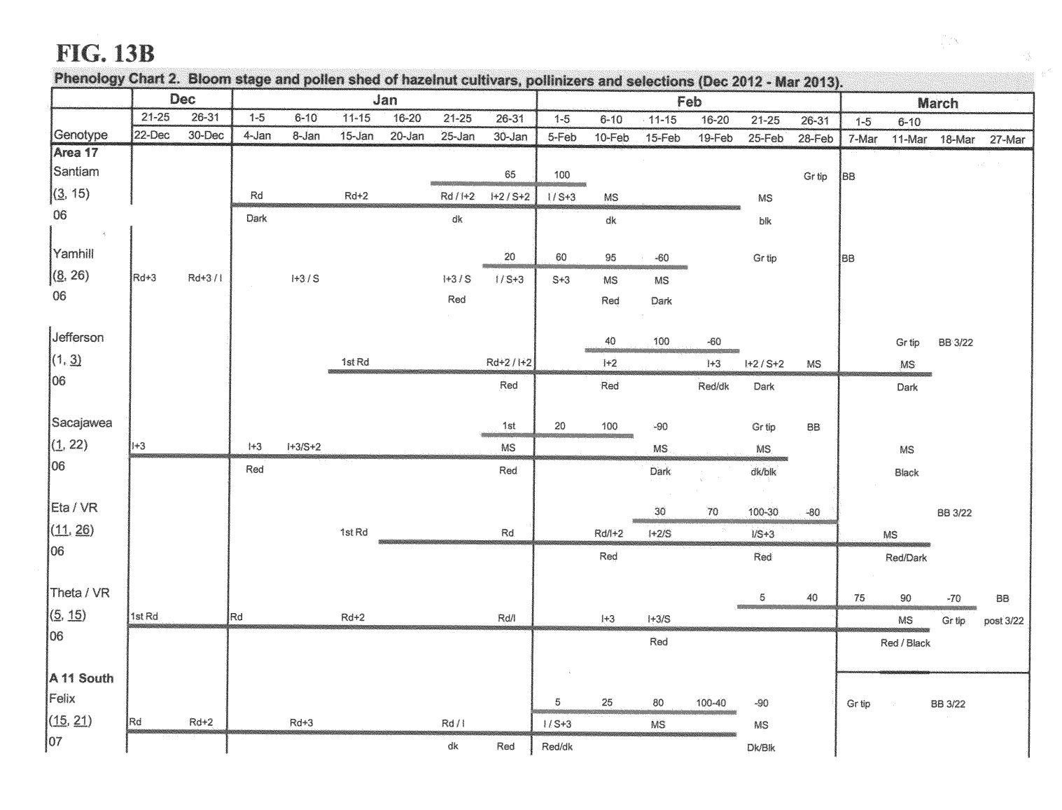

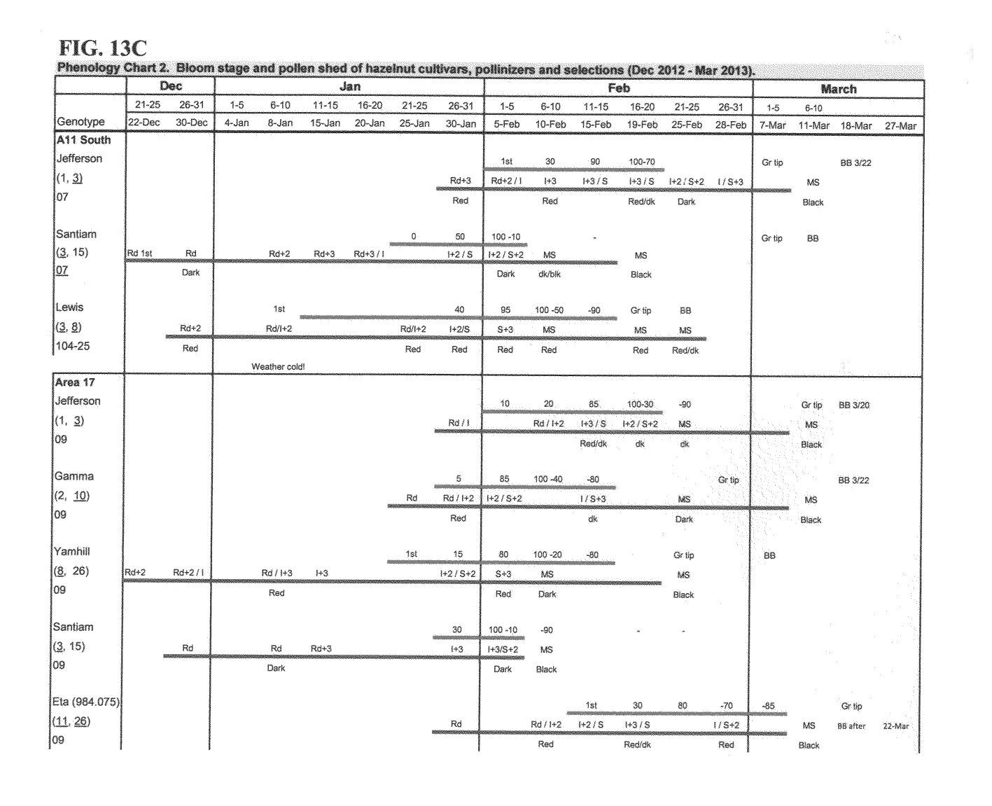

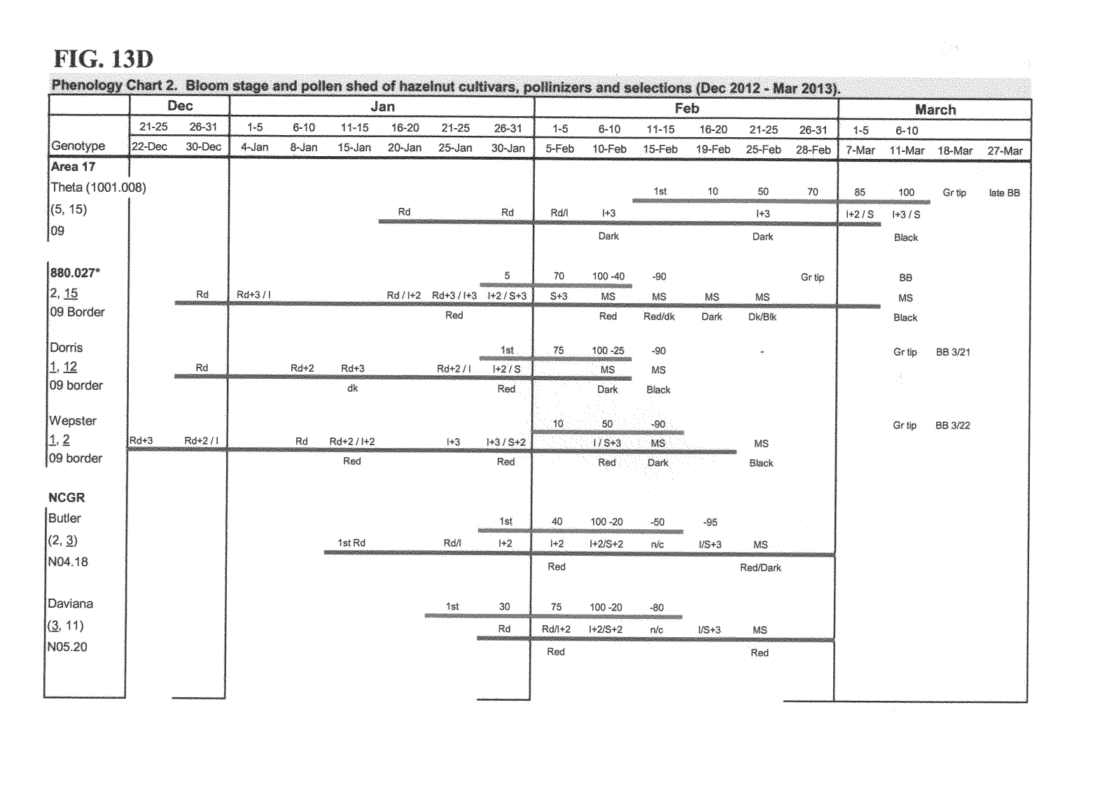

Time of pollen shed and female receptivity were recorded weekly from early December 2011 to late March 2013 (FIGS. 12A-12B). Female inflorescences of ‘McDonald’ emerged in early mid-season and were fully receptive in mid-January. ‘Wepster’ (S₁ S₂) is recommended as a companion cultivar, as ‘Wepster’ and ‘McDonald’ are cross-compatible in both directions. At least one additional pollinizer that sheds compatible pollen in midseason and late-midseason is recommended. Suitable pollinizers include ‘York’ (S₂ S₂₁) and ‘Gamma’ (S₂ S₁₀). ‘Yamhill’ (S₈ S₂₆) is also a suitable pollinizer and is an attractive option as “temporary trees” in double-density plantings. Pollen of ‘Jefferson’ (S₁ S₃) is compatible on females of OSU 880.027, but the time of pollen shed is later than ideal. Pollen of ‘Dorris’ (S₁ S₁₂), ‘Sacajawea’ (S₁ S₂₂) and ‘Barcelona’ (S₁ S₂) is also compatible on females of OSU 880.027. Pollen of ‘Tonda di Giffoni’ (S₂ S₂₃) is incompatible because it expresses S_{2.} Pollen of ‘Delta’ (S₁ S₁₅), ‘Felix’ (S₁₅ S₂₁) and ‘Theta’ (S₅ S₁₅) is incompatible because of the shared allele S_{15.} Inclusion of ‘Wepster’ as a companion cultivar in the orchard results in no loss in total nut yield. Alternative orchard designs include plantings of different cross-compatible cultivars in adjacent rows. Flowering times will continue to be observed, and pollinizer recommendations adjusted accordingly. Pollinizers with a high level of EFB resistance would eliminate the need for fungicide control in the entire orchard.

Pests and diseases.

Based on DNA marker data, ‘McDonald’, like ‘Wepster’ and ‘Yamhill’, has a very high level of resistance to EFB conferred by a dominant allele from ‘Gasaway’, so fungicide applications are not needed. RAPD markers 152-800 and 268-580 that flank the resistance allele in ‘Gasaway’ are present in ‘McDonald’, and these markers are transmitted to its seedlings. Additional RAPD markers linked to resistance are also present. Trees of ‘McDonald’ have not yet been challenged with the EFB pathogen in glasshouse or structure inoculations. No cankers have been observed on the 11 trees of ‘McDonald’ in the yield trials, while several cankers have been noted on adjacent trees of susceptible genotypes.

Susceptibility to bacterial blight caused by Xanthomonas campestris pv. corylina has not been quantified, but no trees in the two trials were affected.

Susceptibility to big bud mite (primarily Phytoptus avellanae Nal.) was rated in the second trial (Table 3) after leaf fall once per year for five years (December 2009-2013). The scale was from 1 (no blasted buds) to 5 (many blasted buds). The average ratings indicate adequate resistance for ‘McDonald’ (1.8), ‘Jefferson’ (1.2), ‘Felix’ (2.0) and ‘Santiam’ (2.2). Blasted buds are very rare on ‘McDonald’, so chemical applications should not be necessary to control bud mite.

Propagation:

Layers of ‘McDonald’ are moderately vigorous and root well, but have lower vigor and caliper than those of ‘Jefferson’ and ‘Barcelona’.

DNA Fingerprinting:

Primers used are shown in Table 5, and results shown in Table 6.

TABLE 5
Primers and annealing temperatures for the 21 microsatellite marker
loci used to fingerprint ′McDonald′ and other hazelnut cultivars.

| | | | Primers 5′-3′ | | | | | | | | | |
|---|---|---|---|---|---|---|---|---|---|---|---|---|
| | | | (Forward above, | | | | | | | | Ref- | |
| Locus | Repeat motif | Size | Reverse below) | Tₐ | n | He | Ho | PIC | r | LG | erence | Locus |
| A614 | (TC)₁₇(CA)₁₀NNN(CA)₆ | 125- | Hex- | 60 | 14 | 0.85 | 0.85 | 0.84 | 0.00 | 6 | Gurcan | A999614 |
| | | 156 | TGGCAGAGCTTTGTC | | | | | | | | et al. | |
| | | | AGCTT | | | | | | | | 2010a | |
| | | | (SEQ ID NO: 1) | | | | | | | | | |
| | | | R- | | | | | | | | | |
| | | | GCAGTGGAGGATTGC | | | | | | | | | |
| | | | TGACT | | | | | | | | | |
| | | | (SEQ ID NO: 2) | | | | | | | | | |
| A616 | (AC)₁₁ | 136- | Fam- | 60 | 13 | 0.85 | 0.85 | 0.83 | 0.00 | 8 | | A616 |
| | | 162 | CACTCATACCGCAAA | | | | | | | | | |
| | | | CTCCA | | | | | | | | | |
| | | | (SEQ ID NO: 3) | | | | | | | | | |
| | | | R- | | | | | | | | | |
| | | | ATGGCTTTTGCTTCGT | | | | | | | | | |
| | | | TTTG | | | | | | | | | |
| | | | (SEQ ID NO: 4) | | | | | | | | | |
| A640 | (CT)₁₅(CA)₁₃ | 354- | F- | 67 | 11 | 0.80 | 0.73 | 0.77 | 0.04 | 10 | Gurcan | A640 |
| | | 378 | TGCCTCTGCAGTTAG | | | | | | | | et al. | |
| | | | TCATCAAATGTAGG | | | | | | | | 2010a | |
| | | | (SEQ ID NO: 5) | | | | | | | | | |
| | | | Fam- | | | | | | | | | |
| | | | CGCCATATAATTGGG | | | | | | | | | |
| | | | ATGCTTGTTG | | | | | | | | | |
| | | | (SEQ ID NO: 6) | | | | | | | | | |
| B619 | (TC)₂₁ | 146- | Fam- | 60 | 14 | 0.88 | 0.88 | 0.87 | 0.00 | 3 | Gurcan | B619 |
| | | 180 | AGTCGGCTCCCCTTT | | | | | | | | et al. | |
| | | | TCTC | | | | | | | | 2010a | |
| | | | (SEQ ID NO: 7) | | | | | | | | | |
| | | | R- | | | | | | | | | |
| | | | GCGATCTGACCTCAT | | | | | | | | | |
| | | | TTTTG | | | | | | | | | |
| | | | (SEQ ID NO: 8) | | | | | | | | | |
| B634 | (AG)₁₅ | 218- | Hex- | 60 | 9 | 0.76 | 0.76 | 0.73 | 0.00 | 4 | Gurcan | B634 |
| | | 238 | CCTGCATCCAGGACT | | | | | | | | et al. | |
| | | | CATTA | | | | | | | | 2010a | |
| | | | (SEQ ID NO: 9) | | | | | | | | | |
| | | | R- | | | | | | | | | |
| | | | GTGCAGAGGTTGCAC | | | | | | | | | |
| | | | TCAAA | | | | | | | | | |
| | | | (SEQ ID NO: 10) | | | | | | | | | |
| B671 | (AG)₆NN(GA)₁₇ | 221- | Hex- | 60 | 13 | 0.86 | 0.88 | 0.84 | - | 9 | Gurcan | B671 |
| | | 249 | TTGCCAGTGCATACT | | | | | | 0.01 | | et al. | |
| | | | CTGATG | | | | | | | | 2010a | |
| | | | (SEQ ID NO: 11) | | | | | | | | | |
| | | | R- | | | | | | | | | |
| | | | ACCAGCTCTGGGCTT | | | | | | | | | |
| | | | AACAC | | | | | | | | | |
| | | | (SEQ ID NO: 12) | | | | | | | | | |
| B709 | (GA)₂₁ | 219- | Ned- | 60 | 8 | 0.74 | 0.76 | 0.70 | - | 5 | Gurcan | B709 |
| | | 233 | CCAAGCACGAATGAA | | | | | | 0.01 | | et al. | |
| | | | CTCAA | | | | | | | | 2010a | |
| | | | (SEQ ID NO: 13) | | | | | | | | | |
| | | | R- | | | | | | | | | |
| | | | GCGGGTTCTCGTTGT | | | | | | | | | |
| | | | ACACT | | | | | | | | | |
| | | | (SEQ ID NO: 14) | | | | | | | | | |
| B733 | (TC)₁₅ | 161- | Ned- | 60 | 8 | 0.68 | 0.68 | 0.63 | 0.00 | 7,2 | Gurcan | B733 |
| | | 183 | CACCCTCTTCACCAC | | | | | | | | et al. | |
| | | | CTCAT | | | | | | | | 2010a | |
| | | | (SEQ ID NO: 15) | | | | | | | | | |
| | | | R- | | | | | | | | | |
| | | | CATCCCCTGTTGGAG | | | | | | | | | |
| | | | TTTTC | | | | | | | | | |
| | | | (SEQ ID NO: 16) | | | | | | | | | |
| B741 | (GT)₅(GA)₁₂ | 176- | Fam- | 60 | 10 | 0.77 | 0.78 | 0.74 | 0.00 | 5 | Gurcan | B741 |
| | | 194 | GTTCACAGGCTGTTG | | | | | | | | et al. | |
| | | | GGTTT | | | | | | | | 2010a | |
| | | | (SEQ ID NO: 17) | | | | | | | | | |
| | | | R- | | | | | | | | | |
| | | | CGTGTTGCTCATGTG | | | | | | | | | |
| | | | TTGTG | | | | | | | | | |
| | | | (SEQ ID NO: 18) | | | | | | | | | |
| B749 | (TC)₁₂ | 200- | Hex- | 60 | 6 | 0.60 | 0.64 | 0.51 | - | I | Gurcan | B749 |
| | | 210 | GGCTGACAACACAGC | | | | | | 0.03 | | et al. | |
| | | | AGAAA | | | | | | | | 2010a | |
| | | | (SEQ ID NO: 19) | | | | | | | | | |
| | | | R- | | | | | | | | | |
| | | | TCGGCTAGGGTTAGG | | | | | | | | | |
| | | | GTTTT | | | | | | | | | |
| | | | (SEQ ID NO: 20) | | | | | | | | | |
| B767 | (TC)₁₅(AT)₇ | 198- | Fam- | 60 | 16 | 0.87 | 0.80 | 0.86 | 0.04 | 8 | Gurcan | B767 |
| | | 238 | CCACCAACTGTTTCA | | | | | | | | et al. | |
| | | | CACCA | | | | | | | | 2010a | |
| | | | (SEQ ID NO: 21) | | | | | | | | | |
| | | | R- | | | | | | | | | |
| | | | GCGAAATGGAGCTCT | | | | | | | | | |
| | | | TGAAC | | | | | | | | | |
| | | | (SEQ ID NO: 22) | | | | | | | | | |
| B774 | (AG)₁₅ | 195- | Ned- | 60 | 8 | 0.80 | 0.80 | 0.77 | 0.01 | 5 | Gurcan | B774 |
| | | 213 | GTTTTGCGAGCTCAT | | | | | | | | et al. | |
| | | | TGTCA | | | | | | | | 2010a | |
| | | | (SEQ ID NO: 23) | | | | | | | | | |
| | | | R- | | | | | | | | | |
| | | | TGTGTGTGGTCTGTA | | | | | | | | | |
| | | | GGCACT | | | | | | | | | |
| | | | (SEQ ID NO: 24) | | | | | | | | | |
| B795 | (TC)₈Ns(CT)₇Ns(CT)₁₀ | 296- | Fam- | 60 | 12 | 0.76 | 0.74 | 0.74 | 0.01 | 9 | Gurcan | B795 |
| | Ns(TC)₅ | 332 | GACCCACAAACAATA | | | | | | | | et al. | |
| | | | ACCTATCTC | | | | | | | | 2010a | |
| | | | (SEQ ID NO: 25) | | | | | | | | | |
| | | | R- | | | | | | | | | |
| | | | TGGGCATCATCCAGG | | | | | | | | | |
| | | | TCTA | | | | | | | | | |
| | | | (SEQ ID NO: 26) | | | | | | | | | |
| C115 | (TAA)₅(GAA)₁₂ | 167- | Fam- | 60 | 14 | 0.80 | 0.80 | 0.77 | 0.00 | 4 | Bassil et | C115 |
| | | 226 | ATTTTCCGCAGATAA | | | | | | | | al. 2005b, | |
| | | | TACAGG | | | | | | | | Gokirmak et | |
| | | | (SEQ ID NO: 27) | | | | | | | | al. 2009 | |
| | | | GTTTCCAGATCTGCC | | | | | | | | | |
| | | | TCCATATAAT | | | | | | | | | |
| | | | (SEQ ID NO: 28) | | | | | | | | | |
| KG807 | (TAAA)AA(TAAA)₂A | 226- | AAGCAAGAAAGGGA | 54 | 4 | 0.67 | 0.78 | 0.60 | - | 11 | Gurcan and | KG807 |
| | (TAAA)₂ | 248 | TGGT | | | | | | 0.07 | | Mehlenbacher | |
| | | | (SEQ ID NO: 29) | | | | | | | | 2010 | |
| | | | FAM- | | | | | | | | | |
| | | | CTTACAGATAAATGG | | | | | | | | | |
| | | | CTCAAA | | | | | | | | | |
| | | | (SEQ ID NO: 30) | | | | | | | | | |
| KG809 | (AGG)₆ | 333- | Hex- | 55 | 5 | 0.66 | 0.64 | 0.60 | 0.01 | 4 | Gurcan and | KG809 |
| | | 345 | AGGCATCAGTTCATC | | | | | | | | Mehlenbacher | |
| | | | CAA | | | | | | | | 2010 | |
| | | | (SEQ ID NO: 31) | | | | | | | | | |
| | | | F- | | | | | | | | | |
| | | | GGAAGGTGAGAGAA | | | | | | | | | |
| | | | ATCAAGT | | | | | | | | | |
| | | | (SEQ ID NO: 32) | | | | | | | | | |
| KG811 | (GA)₁₇ | 240- | Ned- | 58 | 12 | 0.83 | 0.82 | 0.81 | 0.01 | 2 | Gurcan and | KG811 |
| | | 278 | AAGGCGGCACTCGCT | | | | | | | | Mehlenbacher | |
| | | | CAC | | | | | | | | 2010 | |
| | | | (SEQ ID NO: 33) | | | | | | | | | |
| | | | F- | | | | | | | | | |
| | | | GAACAACTGAAGAC | | | | | | | | | |
| | | | AGCAAAG | | | | | | | | | |
| | | | (SEQ ID NO: 34) | | | | | | | | | |
| KG827 | (CT)₁₃AA(CA)₇ | 264- | Fam- | 67 | 9 | 0.78 | 0.84 | 0.75 | - | 9 | Gurcan and | KG827 |
| | | 282 | AGAACTCCGACTAAT | | | | | | 0.04 | | Mehlenbacher | |
| | | | AATCCTAACCCTTGC | | | | | | | | 2010 | |
| | | | (SEQ ID NO: 35) | | | | | | | | | |
| | | | GAGGGAGCAAGTCA | | | | | | | | | |
| | | | AAGTTGAGAAGAAA | | | | | | | | | |
| | | | (SEQ ID NO: 36) | | | | | | | | | |
| KG830 | (CT)₁₄GTATT(CA)₈ | 279- | Ned- | 67 | 9 | 0.79 | 0.78 | 0.76 | 0.00 | 9 | Gurcan and | KG830 |
| | | 311 | TGGAGGAAGTTTTGA | | | | | | | | Mehlenbacher | |
| | | | ATGGTAGTAGAGGA | | | | | | | | 2010 | |
| | | | (SEQ ID NO: 37) | | | | | | | | | |
| | | | AAAGCAACTCATAGC | | | | | | | | | |
| | | | TGAAGTCCAATCA | | | | | | | | | |
| | | | (SEQ ID NO: 38) | | | | | | | | | |
| Soman- | (AAT)₅ | 193- | Hex- | 54 | 3 | 0.60 | 0.98 | 0.51 | - | NA | unpublished | Soman-G |
| G | | 200 | TGGCGTTGCAACATA | | | | | | 0.27 | | | |
| | | | TTCTC | | | | | | | | | |
| | | | (SEQ ID NO: 39) | | | | | | | | | |
| (= 856- | | | R- | | | | | | | | | |
| MS1- | | | GCCATCTTTAGAAAG | | | | | | | | | |
| 13) | | | TTCGATACAG | | | | | | | | | |
| | | | (SEQ ID NO: 40) | | | | | | | | | |

Primers fluorescent tags are FAM, HEX and NED
Tₐ annealing temperature (° C.)
n number of alleles
He expected heterozygosity
Ho observed heterozygosity
PIC polymorphism information content
r estimated null allele frequency
LG linkage group; NA = not yet assigned
Reference for development and characterization

TABLE 6
Allele sizes in ‘McDonald’ and eight other hazelnut cultivars at 21
microsatellite loci.

| | | | | ‘Tonda |
|---|---|---|---|---|
| | | | | Gentile |
| | | ‘Tonda | | delle |
| Locus | ‘McDonald’ | Pacifica’ | ‘Santiam’ | Langhe’ |
| A614 | 135/158 | 135/150 | 132/158 | 125/135 |
| A616 | 150/160 | 150/160 | 150/152 | 150/152 |
| A640 | 362/368 | 368/374 | 355/362 | 355/368 |
| B619 | 158/172 | 166/172 | 158/166 | 150/166 |
| B634 | 222/228 | 228/228 | 222/236 | 228/228 |
| B657 | 211/219 | 211/229 | 219/227 | 219/227 |
| B671 | 229/237 | 229/239 | 225/237 | 239/243 |
| B709 | 229/229 | 229/235 | 229/229 | 229/229 |
| B733 | 173/175 | 173/175 | 175/181 | 173/175 |
| B741 | 178/188 | 178/186 | 186/188 | 176/184 |
| B749 | 207/209 | 207/209 | 209/209 | 207/209 |
| B767 | 200/214 | 200/218 | 212/214 | 214/218 |
| B774 | 203/213 | 203/207 | 209/213 | 203/211 |
| B795 | 317/333 | 315/333 | 317/333 | 315/333 |
| C115 | 174/197 | 174/183 | 194/197 | 174/174 |
| KG807 | 252/252 | 228/252 | 242/252 | 238/252 |
| KG809 | 339/339 | 339/342 | 339/342 | 339/342 |
| KG811 | 245/267 | 245/257 | 257/267 | 257/267 |
| KG827 | 272/284 | 270/284 | 272/272 | 268/278 |
| KG830 | 291/295 | 291/295 | 291/295 | 291/295 |
| Soman-G | 196/200 | 196/200 | 196/200 | 196/200 |
| Locus | ‘Barcelona’ | ‘Wepster’ | ‘Yamhill’ | ‘Dorris’ |
| A614 | 125/132 | 135/158 | 132/158 | 132/158 |
| A616 | 144/152 | 152/160 | 150/150 | 150/152 |
| A640 | 355/374 | 368/374 | 355/368 | 372/374 |
| B619 | 158/172 | 166/172 | 158/172 | 158/166 |
| B634 | 228/228 | 228/228 | 236/236 | 228/228 |
| B657 | 219/223 | 227/229 | 219/229 | 211/227 |
| B671 | 225/229 | 239/249 | 225/243 | 229/249 |
| B709 | 227/235 | 229/235 | 229/229 | 229/229 |
| B733 | 173/175 | 173/175 | 181/185 | 173/181 |
| B741 | 178/186 | 178/186 | 178/186 | 178/186 |
| B749 | 209/209 | 207/209 | 209/209 | 207/207 |
| B767 | 214/240 | 200/242 | 214/238 | 214/218 |
| B774 | 203/207 | 203/207 | 203/211 | 203/207 |
| B795 | 333/333 | 333/333 | 333/333 | 333/333 |
| C115 | 174/194 | 183/194 | 197/216 | 194/216 |
| KG807 | 238/252 | 252/252 | 230/252 | 242/252 |
| KG809 | 339/339 | 342/342 | 348/348 | 339/348 |
| KG811 | 261/267 | 257/257 | 251/261 | 257/267 |
| KG827 | 282/284 | 270/282 | 268/282 | 272/284 |
| KG830 | 291/295 | 295/305 | 291/295 | 295/297 |
| Soman-G | 196/200 | 196/200 | 196/200 | 196/200 |

| Locus | ‘York’ | ‘Felix’ | ‘Gasaway’ |
|---|---|---|---|
| A614 | 124/158 | 138/143 | 143/158 |
| A616 | 144/152 | 150/152 | 150/150 |
| A640 | 363/374 | 368/372 | 362/368 |
| B619 | 158/166 | 158/166 | 172/176 |
| B634 | 228/236 | 228/236 | 222/234 |
| B657 | 221/223 | 219/227 | 225/229 |
| B671 | 243/249 | 229/237 | 237/249 |
| B709 | 229/233 | 229/233 | 229/229 |
| B733 | 173/181 | 175/181 | 175/175 |
| B741 | 178/186 | 186/186 | 186/188 |
| B749 | 209/209 | 207/207 | 207/209 |
| B767 | 236/238 | 214/214 | 214/214 |
| B774 | 203/209 | 203/213 | 203/209 |
| B795 | 333/333 | 321/333 | 317/319 |
| C115 | 197/197 | 197/216 | 216/219 |
| KG807 | 242/252 | 238/242 | 242/252 |
| KG809 | 339/348 | 339/348 | 339/348 |
| KG811 | 257/257 | 251/267 | 257/261 |
| KG827 | 268/272 | 272/284 | 272/282 |
| KG830 | 295/295 | 293/303 | 291/305 |
| Soman-G | 196/200 | 196/200 | 196/196 |

‘McDonald’ fingerprint same as both parents: 2
‘McDonald’ fingerprint same as one parent: 5
‘McDonald’ fingerprint different from both parents: 14

### REFERENCES

Bassil N. V., Botta R., Mehlenbacher S. A. 2005a. Microsatellite markers in hazelnut: Isolation, characterization and cross-species amplification. J. Amer. Soc. Hort. Sci. 130:543-549.
Bassil N. V., Botta R., Mehlenbacher S. A. 2005b. Additional microsatellite markers of the European hazelnut. Acta Hort. 686:105-110.
Bassil N., Boccacci P., Botta R., Postman J. and Mehlenbacher S. A. 2012. Nuclear and chloroplast microsatellite markers to assess genetic diversity and evolution in hazelnut species, hybrids and cultivars. Genetic Resources and Crop Evolution (on-line) DOI 10.1007/s 10722-012-9857-z
Boccacci P., Akkak A., Bassil N. V., Mehlenbacher S. A., Botta R. 2005. Characterization and evaluation of microsatellite loci in European hazelnut (C. avellana) and their transferability to other Corylus species. Molec. Ecol. Notes 5:934-937.
Boccacci P., Akkak, A. and Botta, R. 2006. DNA typing and genetic relations among European hazelnut (Corylus avellana L.) cultivars using microsatellite markers. Genome 49:598-611.
Gökirmak T., Mehlenbacher S. A., Bassil N. V. 2009. Characterization of European hazelnut (Corylus avellana) cultivars using SSR markers. Genetic Resources and Crop Evolution 56:147-172.
G\ddot{r}can, K. and S. A. Mehlenbacher. 2010. Transferability of microsatellite markers in the Betulaceae. J. Amer. Soc. Hort. Sci. 135:159-173.
G\ddot{r}can, K. and S. A. Mehlenbacher. 2010. Development of microsatellite marker loci for European hazelnut (Corylus avellana L.) from ISSR fragments. Molecular Breeding 26:551-559. (available on-line as DOI 10.1007/s11032-010-9464-7)
Gürcan, K., S. A. Mehlenbacher and V. Erdogan. 2010a. Genetic diversity in hazelnut cultivars from Black Sea countries assessed using SSR markers. Plant Breeding 129:422-434. (available on-line doi :10. 1111/j. 1439-0523. 2009. 01753. x).
Gürcan, K., S. A. Mehlenbacher, R. Botta and P. Boccacci. 2010b. Development, characterization, segregation, and mapping of microsatellite markers for European hazelnut (Corylus avellana L.) from enriched genomic libraries and usefulness in genetic diversity studies. Tree Genetics and Genomes 6:513-531. (available on-line as DOI: 10.1007/s11295-010-0269-y)
Mehlenbacher, S. A., D. C. Smith, R. L. McCluskey and M. M. Thompson. 2011. ‘Tonda Pacifica’ hazelnut. HortScience 46:505-508.
Mehlenbacher, S. A., A. N. Azarenko, D. C. Smith, and R. L. McCluskey. 2007. ‘Santiam’ hazelnut. HortScience 42:715-717.
Sathuvalli, V. R. and S. A. Mehlenbacher. 2012. Characterization of American hazelnut (Corylus americana) accessions and Corylus americana×Corylus avellana hybrids using microsatellite markers. Genetic Resources and Crop Evolution 59:1055-1075. DOI 10.1007/s10722-011-9743-0.

We claim:

1. A new and distinct cultivar of Corylus plant as herein illustrated and described.


SEQUENCE LISTING (40 sequences)

SEQ ID NO: 1 - DNA, 20 bp, Artificial Sequence
  Synthetic oligonucleotide
tggcagagctttgtcagctt

SEQ ID NO: 2 - DNA, 20 bp, Artificial Sequence
  Synthetic oligonucleotide
gcagtggaggattgctgact

SEQ ID NO: 3 - DNA, 20 bp, Artificial Sequence
  Synthetic oligonucleotide
cactcataccgcaaactcca

SEQ ID NO: 4 - DNA, 20 bp, Artificial Sequence
  Synthetic oligonucleotide
atggcttttgcttcgttttg

SEQ ID NO: 5 - DNA, 29 bp, Artificial Sequence
  Synthetic oligonucleotide
tgcctctgcagttagtcatcaaatgtagg

SEQ ID NO: 6 - DNA, 25 bp, Artificial Sequence
  Synthetic oligonucleotide
cgccatataattgggatgcttgttg

SEQ ID NO: 7 - DNA, 19 bp, Artificial Sequence
  Synthetic oligonucleotide
agtcggctccccttttctc

SEQ ID NO: 8 - DNA, 20 bp, Artificial Sequence
  Synthetic oligonucleotide
gcgatctgacctcatttttg

SEQ ID NO: 9 - DNA, 20 bp, Artificial Sequence
  Synthetic oligonucleotide
cctgcatccaggactcatta

SEQ ID NO: 10 - DNA, 20 bp, Artificial Sequence
  Synthetic oligonucleotide
gtgcagaggttgcactcaaa

SEQ ID NO: 11 - DNA, 21 bp, Artificial Sequence
  Synthetic oligonucleotide
ttgccagtgcatactctgatg

SEQ ID NO: 12 - DNA, 20 bp, Artificial Sequence
  Synthetic oligonucleotide
accagctctgggcttaacac

SEQ ID NO: 13 - DNA, 20 bp, Artificial Sequence
  Synthetic oligonucleotide
ccaagcacgaatgaactcaa

SEQ ID NO: 14 - DNA, 20 bp, Artificial Sequence
  Synthetic oligonucleotide
gcgggttctcgttgtacact

SEQ ID NO: 15 - DNA, 20 bp, Artificial Sequence
  Synthetic oligonucleotide
caccctcttcaccacctcat

SEQ ID NO: 16 - DNA, 20 bp, Artificial Sequence
  Synthetic oligonucleotide
catcccctgttggagttttc

SEQ ID NO: 17 - DNA, 20 bp, Artificial Sequence
  Synthetic oligonucleotide
gttcacaggctgttgggttt

SEQ ID NO: 18 - DNA, 20 bp, Artificial Sequence
  Synthetic oligonucleotide
cgtgttgctcatgtgttgtg

SEQ ID NO: 19 - DNA, 20 bp, Artificial Sequence
  Synthetic oligonucleotide
ggctgacaacacagcagaaa

SEQ ID NO: 20 - DNA, 20 bp, Artificial Sequence
  Synthetic oligonucleotide
tcggctagggttagggtttt

SEQ ID NO: 21 - DNA, 20 bp, Artificial Sequence
  Synthetic oligonucleotide
ccaccaactgtttcacacca

SEQ ID NO: 22 - DNA, 20 bp, Artificial Sequence
  Synthetic oligonucleotide
gcgaaatggagctcttgaac

SEQ ID NO: 23 - DNA, 20 bp, Artificial Sequence
  Synthetic oligonucleotide
gttttgcgagctcattgtca

SEQ ID NO: 24 - DNA, 21 bp, Artificial Sequence
  Synthetic oligonucleotide
tgtgtgtggtctgtaggcact

SEQ ID NO: 25 - DNA, 24 bp, Artificial Sequence
  Synthetic oligonucleotide
gacccacaaacaataacctatctc

SEQ ID NO: 26 - DNA, 19 bp, Artificial Sequence
  Synthetic oligonucleotide
tgggcatcatccaggtcta

SEQ ID NO: 27 - DNA, 21 bp, Artificial Sequence
  Synthetic oligonucleotide
attttccgcagataatacagg

SEQ ID NO: 28 - DNA, 25 bp, Artificial Sequence
  Synthetic oligonucleotide
gtttccagatctgcctccatataat

SEQ ID NO: 29 - DNA, 18 bp, Artificial Sequence
  Synthetic oligonucleotide
aagcaagaaagggatggt

SEQ ID NO: 30 - DNA, 21 bp, Artificial Sequence
  Synthetic oligonucleotide
cttacagataaatggctcaaa

SEQ ID NO: 31 - DNA, 18 bp, Artificial Sequence
  Synthetic oligonucleotide
aggcatcagttcatccaa

SEQ ID NO: 32 - DNA, 21 bp, Artificial Sequence
  Synthetic oligonucleotide
ggaaggtgagagaaatcaagt

SEQ ID NO: 33 - DNA, 18 bp, Artificial Sequence
  Synthetic oligonucleotide
aaggcggcactcgctcac

SEQ ID NO: 34 - DNA, 21 bp, Artificial Sequence
  Synthetic oligonucleotide
gaacaactgaagacagcaaag

SEQ ID NO: 35 - DNA, 30 bp, Artificial Sequence
  Synthetic oligonucleotide
agaactccgactaataatcctaacccttgc

SEQ ID NO: 36 - DNA, 28 bp, Artificial Sequence
  Synthetic oligonucleotide
gagggagcaagtcaaagttgagaagaaa

SEQ ID NO: 37 - DNA, 29 bp, Artificial Sequence
  Synthetic oligonucleotide
tggaggaagttttgaatggtagtagagga

SEQ ID NO: 38 - DNA, 28 bp, Artificial Sequence
  Synthetic oligonucleotide
aaagcaactcatagctgaagtccaatca

SEQ ID NO: 39 - DNA, 20 bp, Artificial Sequence
  Synthetic oligonucleotide
tggcgttgcaacatattctc

SEQ ID NO: 40 - DNA, 25 bp, Artificial Sequence
  Synthetic oligonucleotide
gccatctttagaaagttcgatacag